(19) United States
(12) Patent Application Publication
(10) Pub. No.: US 2004/0147945 A1
(43) Pub. Date: Jul. 29, 2004

(54) Ultrasonic instrument

(76) Inventor: Gernod Fritzsch, Tuttlingen (DE)

Correspondence Address: SIERRA PATENT GROUP, LTD., P O BOX 6149, STATELINE, NV, 89449, US

(21) Appl. No.: 10/658,103

(22) Filed: Sep. 8, 2003

(30) Foreign Application Priority Data
Sep. 9, 2002 (DE) 102- 41 702.4

(51) Int. Cl. A61B 17/32

(52) U.S. Cl. 606/169

(57) ABSTRACT

A surgical ultrasonic instrument has a housing with an ultrasonic transducer, with a shaft being connected to the housing with an application arrangement being provided at its distal end. The application arrangement includes a working element driven in an oscillating manner by the ultrasonic transducer and a fixed position working element between which a working space is formed for the reception of tissue.

The present invention relates to a surgical ultrasonic instrument comprising a housing which has an ultrasonic transducer and comprising a shaft section which is connected to the housing and at whose distal end an application arrangement is provided, with the application arrangement having a working element driven in an oscillating manner in the axial direction of the shaft by the ultrasonic transducer and a working element in a fixed position in the axial direction.

Surgical ultrasonic instruments of this kind are generally known as so-called ultrasonic scissors and have proven themselves in the past years for the division and preparation of tissue. Ultrasonic scissors are used for the division and/or coagulation of tissue in endoscopic and open surgery.

In known ultrasonic scissors an articulately mounted clamp is pressed against a blade which is set into oscillation by an ultrasonic transducer and thereby triggers thermal effects in the tissue. Cutting or coagulation can take place in dependence on the pressure on the tissue and in dependence on the intended application area.

The problem exists with the known ultrasonic scissors that the movable clamp jaw requires a complex construction, which increases the price of the instrument. In particular endoscopic instruments with a relatively thin shaft (e.g. 5 mm) are very difficult in use and mechanically sensitive.

Furthermore, the known ultrasonic scissors can only be disassembled in part and are difficult to clean, which is disadvantageous with respect to re-use.

It is the object of the present invention to provide a surgical ultrasonic instrument which can be manufactured easily and in a cost-favorable manner and which can be cleaned easily.

This object is satisfied by the features of claim 1 and, with an instrument of the initially named kind, in particular in that the fixed position working element is immovably connected to the shaft and in that the fixed position working element and/or the driven working element is/are shaped such that a working space is formed between them which tapers in the proximal direction.

In accordance with the invention, an application is thus provided at the distal end of the shaft which has a gap which narrows in the proximal direction and is in the form of a fork-like opening into which the tissue to be treated can be pushed. The tissue in the tapering gap is pressed against the driven working element, that is the working element oscillating in the axial direction of the instrument, by the pressure which increases constantly on the insertion of the tissue into the fork-like opening, with the tissue coagulating and/or being cut. The working elements are understood in accordance with the invention as those elements which take up tissue between them and bring about cutting or coagulation by working together.

The instrument in accordance with the invention is simple in design, robust, easy to take part and easy to clean. It can be manufactured in a much more cost-favorable manner and be used for many indications.

Advantageous embodiments of the invention are described in the description, in the drawing and in the dependent claims.

In accordance with a first advantageous embodiment, the fixed position working element can be part of a protective sleeve. A particularly simple construction and simultaneously a good cleaning possibility is hereby provided.

In accordance with a further advantageous embodiment, the movable working element emerges from an end face opening provided in the fixed position working element. A compact, slim construction also results by this.

In accordance with a further embodiment of the invention, the fixed position working element can have an end face extending obliquely to the axial direction which can in particular be planar overall. In this embodiment, a cost-favorable manufacture results due to the planar design of the end face. Furthermore, the cutting effect or the coagulating effect can be influenced by the design of the end face of the fixed position working element. For this reason, it can be advantageous to form the end face of the fixed position working element at least partly curved or to provide sections on the end face which are differently inclined and/or curved with respect to the longitudinal axis of the instrument to optimize the pressure with which the tissue is pressed against the movable working element.

In accordance with a further embodiment, the fixed position working element has lateral tapering sections in comparison with the shaft section. The view of the surgeon in the operating region is hereby considerably improved.

In accordance with a further embodiment of the invention, the movable working element is rotationally symmetrical. It can be advantageous to design the movable working element to taper conically in the distal direction, since the height of the inlet gap, which represents the working height, is thereby enlarged.

In accordance with a further embodiment of the invention, one working element can be adjustable about its longitudinal axis relative to the other working element and can have at least two differently designed working surfaces. It is hereby possible to set differently designed working surfaces by rotation of the one working element relative to the other working element. For example, a sharp cutting edge or, however, a coagulation surface can be set, whereby the cutting properties or the coagulation properties can preferably be selected.

In accordance with a further advantageous embodiment of the invention, the movable working element and the fixed position working element are releasably connected to one another and are in particular easily releasable with a few moves of the hand. A particularly easy cleaning of the instrument hereby results.

The present invention will be described in the following purely by way of example with reference to advantageous embodiments and to the enclosed drawings. There are shown:

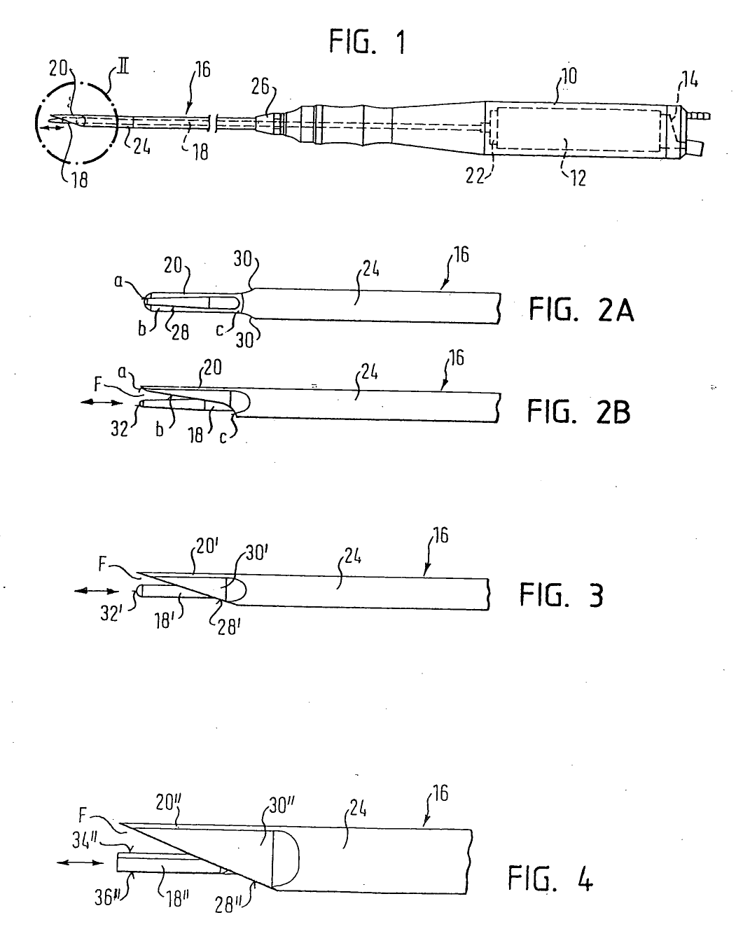

FIG. 1 a schematic side view of a first embodiment of a surgical ultrasonic instrument:

FIG. 2A an enlarged view of the region II of FIG. 1, from below;

FIG. 2B an enlarged view of the region II of FIG. 1;

FIG. 3 a side view of a further embodiment in accordance with the illustration of FIG. 2B; and

FIG. 4 a representation of a further embodiment in accordance with the illustration of FIG. 2B.

The surgical ultrasonic instrument shown in FIG. 1 has a housing 10 in which an ultrasonic transducer 12 is provide which can be supplied with electrical energy via a cable 14 which can be led out of the housing 10. The housing 10 is substantially cylindrical in shape and has a size such that it can easily be grasped by the surgeon's hand. Alternatively, the housing can also have a separate handle and/or actuation switch.

A cylindrical shaft 16 is connected to the distal end of the housing 10 and an application arrangement is provided at its distal end which has a driven working element 18 and a fixed position working element 20 which each have working surfaces facing one another and which are of approximately equal length in the axial direction, i.e. the one working element does not project over the other working element. The driven working element 18 is connected to the ultrasonic transducer 12 via a coupling element 22 and is set into oscillation by the former along the longitudinal axis of the instrument (cf. double arrow in FIG. 1). The fixed position working element 20 is rigidly and immovably connected to the shaft 16 in the embodiment shown. Generally, however, an embodiment can also be considered in which the fixed position working element is admittedly immovably connected to the shaft, but is made flexible overall. In the embodiment shown in FIG. 1, the fixed position working element 20 is part of a protective sleeve 24, that is the fixed position working element 20 is connected in one piece to the protective sleeve 24 which is connected to the housing 2 10 via a screw socket 26.

The movable working element 18 is guided coaxially and inside the protected sleeve 24 and emerges from an end opening provided in the fixed position working element 20 at the distal end of the instrument (cf. FIG. 2A).

As FIGS. 2A and 2B show, the movable working element 18 is rotationally symmetrical and has a dome-shaped, rounded tip 32.

As FIG. 2 further shows, a working space F, which tapers in the proximal direction, is formed by the obliquely extending end face 28 of the fixed position working element 20 between said end face 28 and the driven element 18. In other words, a fork-shaped opening is provided between the fixed position working element 20 and the driven working element 18 and forms a constantly tapering gap.

In the embodiment shown in FIGS. 1-3, the fixed position working element 20 has an end face 28 which has a plurality of differently inclined and curved sections. A convexly curved section a is thus first provided at the foremost distal end of the fixed position working element 20 and is adjoined by a linear section b, i.e. by a planar section, which ultimately merges into a concavely curved section c. In the region of the end face 28, the fixed position working element 20 tapers laterally at both sides—in comparison with the shaft 24— such that lateral tapering sections 30 are formed there in each case which narrow the front end of the shaft 24 in a plan view.

The movable working element 18 of this embodiment tapers conically in the distal direction. The working height of the working space F is hereby enlarged.

FIG. 3 shows, in accordance with FIG. 2B, a side view of a further embodiment of a surgical ultrasonic instrument, with in another respect the components not shown corresponding to the embodiment of FIG. 1.

As FIG. 3 shows, in this embodiment, the fixed position working element 20′ has an end face 28′ which extends obliquely at an angle of approximately 20 to 30° to the longitudinal axis of the instrument and which is planar overall. In a similar manner as with the embodiment of FIG. 2, lateral tapering sections 30′ are provided at the fixed position working element 20′, The movable working element 18′ is circularly cylindrical in shape and likewise has a rounded tip 32′.

FIG. 4 shows a further embodiment of a surgical ultrasonic instrument, with the components not shown corresponding to the embodiment of FIGS. 1-3. In the instrument shown in FIG. 4, the fixed position working element 20″ is made in a similar manner to the embodiment of FIG. 3, that is it has an end face 28″ which is planar overall and from which the movable working element 18″ emerges. The fixed position working element 20″ is likewise provided with tapered sections 30″ at both sides.

The movable working element 18″ is adjustable about its longitudinal axis, that is it can be rotated about its longitudinal axis, whereby differently designed working surfaces 34″ and 36″ can be set. In the position shown in FIG. 4, a knife-shaped or blade-shaped working surface 34″ lies opposite the end face 28″ such that, when tissue is introduced into the working space F, it is pressed against the working surface 34″ by the wedge effect of the working space, whereby a cutting of the tissue takes place. By rotating the driven working element 18″ about 180° about its longitudinal axis, the further working surface 36″ can be set which is flat or planar such that, when tissue is introduced into the working space F, predominantly a coagulation effect is achieved. The rotation of the driven working element 18″ about its longitudinal axis can take place with the aid of a handwheel (not shown) or the like.

When the aforesaid ultrasonic instrument is used, the ultrasonic transducer 12 is supplied with electrical energy via the lead 14, whereby it sets the coupling element 22 and thereby also the driven working element 18 into oscillations which extend parallel to the longitudinal axis of the instrument. By introducing tissue into the working space F between the driven working element and the fixed position working element, the tissue is coagulated and/or cut.

The fixed position working element and the movable working element can each consist of different materials, for example of titanium and of plastic.

### REFERENCE NUMERAL LIST

10 housing

12 ultrasonic transducer

14 lead

16 shaft

18, 18′, 18″ driven working element

20, 20′, 20″ fixed position working element

22 coupling element

24 protective sleeve

26 screw socket

28, 28′, 28″ end face

30, 30′, 30″ tapering section

32, 32′ dome-shaped tip

34″ blade-shaped working surface

36″ flattened working surface

a, b, c sections of the end face

F working space

1. A surgical ultrasonic instrument comprising
a housing (10) which has an ultrasonic transducer (12),
a shaft (16) connected to the housing (10) and having an application arrangement provided at its distal end,
with the application arrangement having a working element (18, 18′, 18″) driven by the ultrasonic transducer (12) in an oscillating manner in the axial direction of the shaft and a working element (20, ‘20’, 20″) with a fixed position in the axial direction, with the fixed position working element (20, 20′, 20″) being immovably connected to the shaft (16), and
the fixed position working element (18, 20; 18″) and the driven working element (20′, 18″, 20″) forming a working space (F) between them which tapers in the proximal direction and which serves for the reception of body tissue in order to coagulate it and/or to cut it.

2. An instrument in accordance with claim 1, characterized in that the fixed position working element (20, 20′, 20″) is part of a protective sleeve (24).

3. An instrument in accordance with claim 1, characterized in that the movable working element (18, 18′, 18″) emerges from an end face opening provided in the fixed position working element (20, 20′, 20″).

4. An instrument in accordance with claim 1, characterized in that the fixed position working element (20, 20′, 20″) has an end face (28, 28′, 28″) extending obliquely to the axial direction and/or has an end face (28, 28″) which is planar overall.

5. An instrument in accordance with claim 1, characterized in that the fixed position working element (20, 20′, 20″) and the movable working element (18, 18′, 18″) are substantially of the same length at the distal end in the axial direction.

6. An instrument in accordance with claim 1, characterized in that the fixed position working element (20′) has an end face (28) which is at least partly curved and/or has an end face (28) which has differently inclined and/or curved sections (a, b, c) relative to the longitudinal axis of the instrument.

7. An instrument in accordance with claim 1, characterized in that the fixed position working element (20, 20′, 20″) tapers laterally in comparison with the shaft (24).

8. An instrument in accordance with claim 1, characterized in that the movable working element (18, 18′) is rotationally symmetrical.

9. An instrument in accordance with claim 1, characterized in that the movable working element (18) tapers conically in the distal direction.

10. An instrument in accordance with claim 1, characterized in that a working element (18″) is adjustable about its longitudinal axis relative to the other working element (20″) and has at least two differently designed working surfaces; and/or in that the movable working element (18, 18′, 18″) and the fixed position working element (20, 20′, 20″) are releasably connected to one another.
